(19) United States
(12) Patent Application Publication
(10) Pub. No.: US 2005/0014647 A1
(43) Pub. Date: Jan. 20, 2005

(54) Method and composition for determining resistance to an acetohydroxy-acid synthase inhibitor

(76) Inventor: Dale L. Shaner, Fort Collins, CO (US)

Correspondence Address: Morrison & Foerster, 1650 Tysons Blvd, Suite 300, McLean, VA, 22102, US

(21) Appl. No.: 10/485,509

(22) PCT Filed: Jul. 27, 2002
(86) PCT No.: PCT/EP02/08390

Related U.S. Application Data
(60) Provisional application No. 60/310,014, filed on Aug. 3, 2001.

(51) Int. Cl. C12Q 1/26; A01N 25/00

(52) U.S. Cl. 504/116.1; 435/25

(57) ABSTRACT

The invention describes a method for determining whether a plant is resistant to an acetohydroxyacid synthase (“AHAS”) inhibitor. The method comprises: (a) treating a part taken from the plant with a acetohydroxyacid synthase inhibitor, a ketol-acid reductoisomerase inhibitor and an external supplement that is either a compound or combination of compounds selected from the group consisting of alanine, ammonium hydroxide, asparagine, and pyruvic acid or a salt thereof; and (b) measuring the amounts of acetohydroxybutyrate and/or acetolactate.

### BACKGROUND OF THE INVENTION

Certain classes of herbicides (e.g., an imidazolinone, sulfonylurea, triazolopyrimidine and pyrimidyl salicylic acid) destroy plants by inhibiting acetohydroxyacid synthase (AHAS), which is also known as acetolactate synthase. AHAS is the first enzyme in the branched chain amino acid pathway.

Crop varieties have been developed that are resistant to AHAS inhibitor herbicides. In addition, overuse of these classes of herbicides has propagated selected weed populations that are resistant to AHAS inhibition.

A need has thus developed to rapidly detect both resistant crops and weed populations. This need led to the discovery of various in vivo AHAS assay methods, e.g. as described in U.S. reissue Pat. No. 36,175 reissued Mar. 30, 1999, U.S. Pat. No. 5,356,789 issued Oct. 18, 1994 and U.S. Pat. No. 5,932,434 issued Aug. 3, 1999, both of which are incorporated herein by reference.

The assays in these references are based on an inhibitor of ketoacid reductoisomerase (KARI), which is the next enzyme after AHAS in the branched chain amino acid pathway. Inhibition of KARI results in the accumulation of acetolactate in the plant. Acetolactate is the product of AHAS. Thus, the in vivo activity of AHAS can be measured by combining a KARI inhibitor with an AHAS inhibitor and measuring the amount of acetolactate in the plant.

These prior art assays have the advantage of being able to obtain an indication of AHAS resistance, without the need for elaborate biochemical expertise or equipment. The disadvantage is that it requires a substantial amount of plant tissue, in order to accumulate enough acetolactate for the detection and quantification of AHAS resistance.

Imidazolinone resistant crop varieties have been developed in several crops including maize, canola, wheat, rice and sunflowers. E.g., please see generally European patent 508,161 granted May 10, 2000 and European patent application 965,265 published Dec. 12, 1999, both of which are incorporated herein by reference. The herbicide resistance is due to a single base pair change in the gene encoding for the AHAS enzyme, so that it no longer binds the herbicide.

In many crops, there is more than one gene that encodes for AHAS. For example, in canola and wheat, the commercially available imidazolinone resistant varieties have two genes. If the AHAS is encoded by only one gene, the level of herbicide resistance at the whole plant level is insufficient for commercial use.

The in vivo AHAS assays can be used to differentiate between plant lines containing various levels of a resistant AHAS enzyme. However, the assays need to be done on relatively large plants, in order to obtain enough plant material to run the assay. Also, the throughput time is relatively long. One way to do this is to spray the plant with a high rate of an imidazolinone. Although this method is very accurate, it requires a relatively large greenhouse and length of time, both of which increase the expense of developing new imidazolinone resistant crop varieties. Also, it limits the amount of plant material that can be handled.

Another way is to conduct in vitro AHAS assays, which tends to be very accurate, but requires access to a fully equipped biochemical laboratory and also takes a considerable amount of time.

For all of the reasons, above, a plant breeder needs a rapid and nondestructive method to determine the level of herbicide resistance in plant breeding lines. This felt need has thus led to this invention, which is a high throughput, in vivo assay method and composition for detecting AHAS resistance.

The method and composition described in the invention increases the rate of accumulation of acetolactate. Thus, the plant tissue requirement is reduced. Also, the assay can take advantage of the microtiter plate system that is currently used for high throughput assays in the pharmaceutical industry.

The objective of this assay is a method for increasing the rate of accumulation of acetolactate in plant material in the presence of a KARI inhibitor, and thus reduce the amount of plant material needed to run the assay to the point where the assay could be run in a microtiter plate. The objective is accomplished by determining that external supplementation of the in vivo AHAS assay medium greatly increases the rate of accumulation of acetolactate in plant tissue.

In one embodiment of the invention, the method for determining whether a plant is resistant to an acetohydroxyacid synthase inhibitor comprises the following steps;
(a) treating a part taken from the plant with an effective amount of the acetohydroxyacid synthase inhibitor, a ketol-acid reductoisomerase inhibitor and an external supplement that is either a compound or combination of compounds selected from the group consisting of alanine, ammonium hydroxide, asparagine, and pyruvic acid or a salt thereof; and
(b) measuring the amounts of acetohydroxybutyrate and/or acetolactate.

In a preferred embodiment, the method according for determining whether a plant is resistant to an acetohydroxyacid synthase inhibitor comprises the following steps:
(a) treating a first part taken from the plant with an effective amount of the acetohydroxyacid synthase inhibitor, a ketol-acid reductoisomerase inhibitor, and an external supplement that is either a compound or combination of compounds selected from the group consisting of alanine, ammonium hydroxide, asparagine, and pyruvic acid or a salt thereof; and
(b) treating a second part taken from the plant with an external supplement that is either a compound or combination of compounds selected from the group consisting of alanine, ammonium hydroxide, asparagine, and pyruvic acid or a salt thereof; or
(c) treating a second part taken from the plant with an effective amount of the ketol-acid reductoisomerase inhibitor and with an external supplement that is either a compound or combination of compounds selected from the group consisting of alanine, ammonium hydroxide, asparagine, and pyruvic acid or a salt thereof; and
(d) determining the amounts of acetohydroxybutyrate and/or acetolactate accumulated in the part of the plant from step a) and in the part of the plant from step b) or c); and
(e) comparing the amount of acetohydroxybutyrate and/or acetolactate from step a) with step b) or c).

The part taken from the plant in the above-mentioned methods can be taken from a blade with a first and second portion that is separated by a midvein. The first part of the plant used in step a) is taken from the first portion of the blade. The second part of the blade used in step b) or c) can be taken from the second portion at an approximately equal and opposite location from the midvein in the blade.

In yet another embodiment, the method according to the present invention for determining whether a population of a plant species is resistant to an acetohydroxyacid synthase inhibitor, the plant species has a blade with a first and second portion that is separated by a midvein. The method comprises:
(a) treating a first part taken from the first portion, with an effective amount of the acetohydroxyacid synthase inhibitor, a ketol-acid reductoisomerase inhibitor, and either a compound or combination of compounds selected from the group consisting of alanine, ammonium hydroxide, asparagine, and pyruvic acid or a salt thereof;
(b) treating a second part taken from the second portion at an approximately equal and opposite location from the midvein in the blade, with an effective amount of the ketol-acid reductoisomerase inhibitor in step (a);
(c) obtaining a first indication in the first part and a second indication in the second part of either or both acetolactate and acetohydroxybutyrate; and
(d) contrasting the first and second indication.

If the method is done in a High Through-Put Screening it is preferable to use only small amounts of the plant tissue. For example, each of the first and second part or of the part of the plant of step a) can be even less than about 50 mg, but also less than about 20 mg and even less than about 10 mg. In a specific embodiment, the first and second portion is within a basipetal region of the blade. In a more specific embodiment, the plant species comprises a leaf having a blade and a petiole. In a yet more specific embodiment, the basipetal region is adjacent to the petiole.

In a further embodiment, the invention is a method for determining whether a test compound inhibits acetohydroxyacid synthase in a plant. All of the above-mentioned embodiments of the above-mentioned methods can be used for this method except that the acetohydroxyacid synthase inhibitor is replaced with a test-compound.

In detail, said methods comprises:
(a) treating a part taken from the plant with a test-compound, a ketol-acid reductoisomerase inhibitor and an external supplement that is either a compound or combination of compounds selected from the group consisting of alanine, ammonium hydroxide, asparagine, and pyruvic acid or a salt thereof; and
(b) measuring the amounts of acetohydroxybutyrate and/or acetolactate.

Numerous salts of pyruvic acid are known by the skilled artisan e.g. the natrium, kalium or potassium salt.

In yet another embodiment the method for determining whether a test-compound inhibits acetohydroxyacid synthase comprises
(a) treating a first part taken from the plant with a test-compound, a ketol-acid reductoisomerase inhibitor, and an external supplement that is either a compound or combination of compounds selected from the group consisting of alanine, ammonium hydroxide, asparagine, and pyruvic acid or a salt thereof; and
(b) treating a second part taken from the plant with an external supplement that is either a compound or combination of compounds selected from the group consisting of alanine, ammonium hydroxide, asparagine, and pyruvic acid or a salt thereof; or
(c) treating a second part taken from the plant with a ketol-acid reductoisomerase inhibitor and with an external supplement that is either a compound or combination of compounds selected from the group consisting of alanine, ammonium hydroxide, asparagine, and pyruvic acid or a salt thereof; and
(d) determining the amounts of acetohydroxybutyrate and/or acetolactate accumulated in the part of the plant from step a) and in the part of the plant from step b) or c); and
(e) comparing the amount of acetohydroxybutyrate and/or acetolactate from step a) with step b) or c).

The part taken from the plant in the above-mentioned methods can be taken from a blade with a first and second portion that is separated by a midvein. The first part of the plant used in step a) is taken from the first portion of the blade. The second part of the blade used in step b) or c) can be taken from the second portion at an approximately equal and opposite location from the midvein in the blade.

All of the above-mentioned methods are herein below termed “methods according to the invention”.

In a preferred embodiment, the part of the plant used in a methods according to the invention is selected from an actively growing part of the plant. Activly growing means that the tissue should be in the logarithmic stage of growth for maximum activity.

Furthermore, it is preferred that the part of the plant used in the methods of the present invention is taken from the basipetal region of a blade from the plant.

The amounts of acetohydroxybutyrate and/or acetolactate can be determined as described U.S. Pat. No. 5,356,789 based on the use creatine and naphthol. The activity can be either determined by measuring OD₅₃₀ or by comparing the intensity of colour visually.

The acetohydroxyacid synthase inhibitor can be selected from certain classes of herbicides that inhibit acetohydroxyacid synthase (e.g., an imidazolinone, sulfonylurea, triazolopyrimidine and pyrimidyl salicylic acid). Numerous examples of these herbicides are well known by the skilled artisian and e.g. given in the Pesticide manual, 12th Edition, 2000 British Crop Protection Councel).

The amount of the acetohydroxyacid synthase inhibitor, a ketol-acid reductoisomerase inhibitor (herein further also referred to as “effective amount”) can be determined easily by the skilled artisian. As a rule, if the plant part has a weight of 50 mg, the effective amount of the acetohydroxyacid synthase inhibitor, a ketol-acid reductoisomerase inhibitor is between 100-500 μM.

The external supplement is either a compound or combination of compounds selected from the group consisting of alanine, ammonium hydroxide, asparagine, and pyruvic acid (or a salt thereof), preferably alanine alone, alanine combined with pyruvic acid (or a salt thereof) or ammonium hydroxide and pyruvic acid (or a salt thereof).

It is preferred to use small leaf discs in the method of the present invention. This is because the smaller the leaf discs, the greater the ratio of edge to total area and the better is the entrance of chemicals into the leaf.

Preferably, the external supplement in step a) of the methods according to the invention, has an amount of about 0.2 to less than 5 percent by weight of a sum consisting of the part taken from the plant, the effective amount of the acetohydroxyacid synthase inhibitor, the ketol-acid reductoisomerase inhibitor and the external supplement of step a).

Furthermore, it has been found that certain combinations of the external supplements show a synergistic increase in accumulation of acetolactate in the assays according to the invention.

In a preferred embodiment, the supplement used in methods according to the invention is the combination of compounds alanine and pyruvic acid or a salt thereof. In a specific embodiment, the alanine and pyruvic acid or salt is in a ratio of about 8:1 to 1:50. In another specific embodiment, the alanine, and pyruvic acid or salt has an amount of about 0.2 to less than 5 percent by weight of a sum consisting of the first part, the acetohydroxyacid synthase and ketol-acid reductoisomerase inhibitor, and the alanine, and pyruvic acid or salt. In a more specific embodiment, the alanine is L-alanine.

In still another preferred embodiment, the supplement used in methods mentioned above the combination of compound ammonium hydroxide, and pyruvic acid or a salt thereof. In a specific embodiment, the ammonium hydroxide and pyruvic acid or salt is in a mole to mole ratio of about 8:1 to about 1:50. In another specific embodiment, the ammonium hydroxide, and pyruvic acid or salt has an amount of about 0.2 to less than 5 percent by weight of a sum in step (a) consisting of the first part, the acetohydroxyacid synthase and ketol-acid reductoisomerase inhibitor, and the ammonium hydroxide, and pyruvic acid or salt.

In yet another preferred embodiment, the method for determining whether a plant is resistant to a herbicide comprises:
(a) combining in an aqueous medium a fresh sample of a tissue from the plant, the fresh sample is an amount of less than about 50 mg, an effective amount of the herbicide, an effective amount of a ketol-acid reductoisomerase inhibitor, and either a compound or a combination of compounds selected from the group consisting of alanine, ammonium hydroxide, asparagine, and pyruvic acid or a salt thereof, the compound or combination of compounds is an amount of about 0.2 to less than 5 percent by weight of a sum of the fresh sample and herbicide, the ketol-acid reductoisomerase inhibitor, and the compound or combination of compounds; and
(b) identifying an accumulation of acetolactate.

In yet another embodiment, the invention is a composition comprising alanine, and pyruvic acid or a salt thereof. In a specific embodiment, the alanine, and pyruvic acid or salt are in a mole to mole ratio of 8:1 to about 1:50. In another specific embodiment, the composition is in an aqueous medium.

In still yet another embodiment, the invention is a composition comprising ammonium hydroxide, and pyruvic acid or a salt thereof. In a specific embodiment, the ammonium hydroxide, and pyruvic acid or salt are in a mole to mole ratio of 8:1 to about 1:50. In another specific embodiment, the composition is in an aqueous medium.

In a further embodiment, the imethod for determining whether a material inhibits acetohydroxyacid synthase in a plant comprises:
(a) treating a first part taken from the first portion, with an effective amount of the material, a ketol-acid reductoisomerase inhibitor, and either a compound or combination of compounds selected from the group consisting of alanine, ammonium hydroxide, asparagine, and pyruvic acid or a salt thereof;
(b) treating a second part taken from the second portion at an approximately equal and opposite location from the midvein in the blade, with an effective amount of the ketol-acid reductoisomerase inhibitor in step (a); and
(c) obtaining a first indication in the first part and a second indication in the second part of either or both acetolactate and acetohydroxybutyrate; and
(d) contrasting the first and second indication, to determine if an amount of either or both acetolactate and acetohydroxybutyrate present in the first indication is less than an amount of either or both acetolactate and acetohydroxybutyrate present in the second indication.

The term “indication” means determination of the level of either or both acetolactate and acetohydroxybutyrate. Methods how to determine this indication are disclosed above.

In a more specific embodiment, each of the first and second part is taken from a basipetal region of the blade.

In a still further embodiment, the invention is a method for determining whether a herbicide is capable of inhibiting acetolactate synthesis in a plant. The method comprises:
(a) combining in an aqueous medium a fresh sample of a tissue from the plant, the fresh sample being an amount of less than about 50 mg, and an effective amount of the herbicide, a ketol-acid reductoisomerase inhibitor, and either a compound or a combination of compounds selected from the group consisting of alanine, ammonium hydroxide, asparagine, and pyruvic acid or a salt thereof, the compound or combination of compounds being from about 0.2 to less than 5 percent by weight of a sum of the fresh sample and herbicide, the ketol-acid reductoisomerase inhibitor, and the compound or combination of compounds; and
(b) determining if there is an accumulation of acetolactate from step (a).

A ketol-acid reductoisomerase inhibitor described in all of the embodiments above, is disclosed in the prior art. In one embodiment, the ketol-acid reductoisomerase inhibitor is selected from the group consisting of (dimethylphosphinyl) glycolic acid, 2-(dimethylphosphinoyl)-2-hydroxyacetic acid, a sodium N-hydroxy-N-(C₁-C₆ alkyl or C₃-C₇ cycloalkyl)oxamate, sodium N-hydroxy-N-aralkyloxamate, 2-methylphosphinoyl-2-hydroxyacetic acid, N-hydroxy-N-isopropyloxamate, the monomethyl and monoethyl ester of 1,1-cyclopropanedicarboxylic acid, and ethylenemalonic acid and the mono and disalt therof. In a specific embodiment, the sodium N-hydroxy-N-(C₁-C₆ alkyl) oxamate is sodium N-hydroxy-N-isopropyloxamate.

In another specific embodiment, the sodium N-hydroxy-N-aralkyloxamate is sodium N-hydroxy-N-benzyloxamate.

The increased accumulation of acetolactate was used to reduce the plant material to amounts that leads to small samples enabeling high throughput screening. For example, a 5 mm diameter leaf disc that could easily fit in a 96-well microtiter plate well can be used in the method according to the present invention. Using high throughput screening, many discrete compounds can be tested n parallel so that large numbers of test compounds can be quickly screened.

The most widely established techniques utilize 96-well, 384-well and 1536-well microtiter plates. As mentioned above, the 96-well formate is preferred in the above-mentioned applicaiton. In addition to the plates, many instruments, materials, pipettors, robotics, plate washers, and plate readers are commercially available to fit the respective well format.

In addition, a method was developed for cutting leaf discs from plant material and for extracting the acetolactate from the leaf discs in situ in the microtiter plate so that the assay can easily be run on over 1000 plants per week.

Furthermore, if freeze thawing is used for tissue extraction, it is also possible that the plates can be stored frozen for an extended period of time before complementing the rest of the reaction.

### EXAMPLES

The procedure for the preparation of an in vivo AHAS assay is described below:

For the experiments, plants are grown from seed in soil in a greenhouse. Most experiments used wild type and imidazolinone resistant varieties of wheat, canola, rice, sunflower, and maize.

For the assay, a a microtiter plate is set up with 100 μl of one of the following solutions in each well:
a. Solution A: 10 mM phosphate buffer, pH 6; 10% Murashige and Skoog basal mineral salts (according to Murashige, T. and Skoog, F., Physiol. Plant., 15 473-497 (1962)); 500 μM CPCA (cyclopropyldicarboxylic acid);
b. Solution B: Solution A+alanine (or other supplements);
c. Solution C: Solution A+a pyruvate salt (or other supplements).

Plant material is excised from the newest emerging, rapidly growing leaf or stem from the test plant and placed in one of the microtiter plates. Usually, 5 mm leaf discs are cut from the newest emerging leaf. At least 2 discs per plant are cut. One disc is put in a well with Solution A and the other disc in a well with one of the other Solutions B or C (what solution are you using for background?).

After all wells in the microtiter plate contain plant material, the plate is covered with the microtiter plate cover and the plate with material is placed in a chamber with continuous fluorescent lights for 12 to 16 hours. The incubation is stopped by placing the plate in −20° C. freezer. After tissue and solutions freeze, the plate is thawed at room temperature and 25 μL of 5% sulfuric acid are added to each well. After incubation at 60° C. for 15 minutes, 175 μL of a freshly prepared mixture of α-naphthol (2.5%) and creatine (0.25%) in 2 N NaOH are added to each well and the plate is incubated at 60° C. for 15 minutes.

200 μL from each well are transferred to a new plate and OD₅₃₀ on microtiter plate is readed by a spectrophotometer.

The results of the in vivo AHAS assays are shown in the tables, below.

Table 1

Effect of L-alanine on in vivo AHAS activity in leaf discs from newest emerging leaf of different species. Incubated in assay solution for 14 hours. (All assay solutions contained 500 μM CPCA, and 10% Murashige and Skoog mineral salts.)

(Ratio indicates the amount of increased AHAS activity in the presence of L-alanine)

| | |
|---|---|
| OD 530 | |
| Alanine Concentration (%) | Ratio |

| Species | 0.000 | 1.000 | (Ala/No Ala) |
|---|---|---|---|
| Maize | 0.120 | 0.203 | 1.7 |
| Wheat | 0.400 | 0.832 | 2.1 |
| Rice | 0.180 | 1.257 | 7.0 |
| Sunflower | 0.490 | 1.857 | 3.8 |
| Canola | 0.523 | 2.838 | 5.4 |

Table 2

Interaction of L-alanine supplementation and pH of incubation solution on in vivo AHAS activity in leaf discs from newest emerging leaf of different species. Incubated in assay solution for 14 hours. (All assay solution contained 10 mM Phosphate buffer adjusted to the different pHs, 500 μM CPCA, and 10% Murashige and Skoog mineral salts.)

| | |
|---|---|
| Alanine (%) | Ratio |

| Species | pH | 0 | 1 | (Ala/No Ala) |
|---|---|---|---|---|
| Canola | 4 | 0.816 | 2.271 | 2.78 |
| | 5 | 0.811 | 2.383 | 2.94 |
| | 6 | 0.653 | 2.036 | 3.12 |
| | 7 | 0.964 | 2.299 | 2.38 |
| Rice | 4 | 0.319 | 0.903 | 2.83 |
| | 5 | 0.205 | 0.731 | 3.57 |
| | 6 | 0.175 | 0.639 | 3.65 |
| | 7 | 0.175 | 0.58 | 3.31 |
| Sunflower | 4 | 0.36 | 1.682 | 4.67 |
| | 5 | 0.206 | 0.745 | 3.62 |
| | 6 | 0.162 | 0.843 | 5.20 |
| | 7 | 0.117 | 0.48 | 4.10 |
| Maize | 4 | 0.48 | 0.909 | 1.89 |
| | 5 | 0.242 | 0.847 | 3.50 |
| | 6 | 0.181 | 1.033 | 5.71 |
| | 7 | 0.261 | 1.128 | 4.32 |

Table 3

Effect of different concentrations of L-alanine on in vivo AHAS activity in leaf discs taken from newest emerging leaf of two canola varieties. (Increased activity based on comparison between no alanine with different levels of alanine.) Incubated in assay solution for 14 hours. (All assay solutions contained 10 mM Phosphate buffer, pH 6, 500 μM CPCA, and 10% Murashige and Skoog mineral salts.)

| | |
|---|---|
| OD 530 | Increased Activity (%) |

| Alanine (%) | Westar | 45A71 | Westar | 45A71 |
|---|---|---|---|---|
| 0 | 0.501 | 0.524 | | |
| 0.063 | 0.978 | 0.785 | 195 | 150 |
| 0.125 | 0.885 | 0.955 | 177 | 182 |
| 0.25 | 1.197 | 0.946 | 239 | 181 |
| 0.5 | 1.149 | 1.038 | 229 | 198 |
| 1 | 1.983 | 1.373 | 396 | 262 |
| 2 | 1.25 | 1.518 | 250 | 290 |
| 4 | 1.009 | 0.997 | 201 | 190 |

Table 4

Effect of taking leaf discs from different parts of a leaf in canola on in vivo AHAS activity. Leaf position denotes from the tip of the leaf toward the base of the leaf. Leaf 4 was the newest emerging leaf and Leaf 3 was the second youngest leaf that was still rapidly expanding. Incubated in assay solution for 14 hours. (All assay solutions contained 10 mM Phosphate buffer, pH 6, 500 μM CPCA, 1% Alanine, 100 mM Pyruvate and 10% Murashige and Skoog mineral salts.)

OD 530

| Leaf Position | Leaf 4 | Leaf 3 |
|---|---|---|
| Tip | 1.065 | 0.672 |
| Middle | 1.542 | 0.741 |
| Base | 2.089 | 1.206 |

Table 5

Effect of different sizes of leaf discs on in vivo AHAS activity in newest emerging leaf in sunflower. The number of discs indicates the total number that was in an incubation solution so that the total area of the discs was approximately the same in each assay. Incubated in assay solution for 14 hours. (All assay solutions contained 10 mM Phosphate buffer, pH 6, 500 μM CPCA, 1% Alanine, and 10% Murashige and Skoog mineral salts.)

| | | Total Area of | |
|---|---|---|---|
| Disc Size | | Discs | |
| (cm²) | Number of Discs | (cm²) | OD 530 |
| 1.76 | 1 | 1.76 | 0.447 |
| 0.82 | 2 | 1.64 | 0.646 |
| 0.56 | 3 | 1.68 | 0.695 |
| 0.25 | 7 | 1.75 | 1.861 |

Table 6

Effect of different combinations of NH₄OH and Na-Pyruvate on in vivo AHAS activity in excised leaf discs of canola. Data are shown as the percentage of activity compared to a paired check with no supplementation of NH₄OH or Na-pyruvate. Incubated in assay solution for 14 hours. (All assay solutions contained 10 mM Phosphate buffer, pH 6, 500 μM CPCA, and 10% Murashige and Skoog mineral salts.)

| | | % of No NH₄ or |
|---|---|---|
| NH₄ (mM) | Pyruvate (mM) | Pyruvate |
| 6.25 | 0 | 101 |
| 12.5 | 0 | 138 |
| 25 | 0 | 128 |
| 50 | 0 | 142 |
| 0 | 6.25 | 109 |
| 0 | 12.5 | 128 |
| 0 | 25 | 138 |
| 0 | 50 | 175 |
| 6.25 | 6.25 | 182 |
| 12.5 | 6.25 | 125 |
| 25 | 6.25 | 162 |
| 50 | 6.25 | 161 |
| 6.25 | 12.5 | 152 |
| 12.5 | 12.5 | 162 |
| 25 | 12.5 | 185 |
| 50 | 12.5 | 161 |
| 6.25 | 25 | 191 |
| 12.5 | 25 | 143 |
| 25 | 25 | 189 |
| 50 | 25 | 164 |
| 6.25 | 50 | 257 |
| 12.5 | 50 | 264 |
| 25 | 50 | 274 |
| 50 | 50 | 211 |

Table 7

Effect of L-alanine, L-asparagine, NH4OH and Na-Pyruvate on in vivo AHAS activity in excised leaf discs from canola. Avg. is the average of the OD530 of the treated discs. Incubated in assay solution for 14 hours. (All assay solutions contained 10 mM Phosphate buffer, pH 6, 500 μM CPCA, and 10% Murashige and Skoog mineral salts.)

### OD 530

Replicate

| | | |
|---|---|---|
| 1 | 2 | 3 |

Alanine (100 mM)

| | | | |
|---|---|---|---|
| 0/0 | 1.193 | 1.518 | 1.301 |
| Treated | 1.529 | 1.487 | 1.335 |
| Treated | 1.760 | 1.691 | 1.551 |
| Treated | 1.498 | 1.571 | 1.007 |
| Avg. | 1.538 | 1.574 | 1.298 |
| % of 0/0 | 129 | 104 | 100 |

Asparagine (100 mM)

| | | | |
|---|---|---|---|
| 0/0 | 1.293 | 0.563 | 1.325 |
| Treated | 1.011 | 0.718 | 1.279 |
| Treated | 1.343 | 0.880 | 1.094 |
| Treated | 1.843 | 0.907 | 1.186 |
| Avg. | 1.384 | 0.796 | 1.206 |
| % of 0/0 | 107 | 141 | 91 |

NH₄OH (50 mM)

| | | | |
|---|---|---|---|
| 0/0 | 0.618 | 0.799 | 0.885 |
| Treated | 0.604 | 0.880 | 1.386 |
| Treated | 0.522 | 0.866 | 1.177 |
| Treated | 1.003 | 0.953 | 1.845 |
| Avg. | 0.697 | 0.886 | 1.386 |
| % of 0/0 | 113 | 111 | 157 |

Pyruvate (100 mM)

| | | | |
|---|---|---|---|
| 0/0 | 1.128 | 1.132 | 1.040 |
| Treated | 1.526 | 1.848 | 1.972 |
| Treated | 1.469 | 1.846 | 2.235 |
| Treated | 1.725 | 1.796 | 2.100 |
| Avg. | 1.510 | 1.730 | 1.950 |
| % of 0/0 | 134 | 153 | 188 |

Pyr & NH₄OH (50 mM each)

| | | | |
|---|---|---|---|
| 0/0 | 0.388 | 0.861 | 0.835 |
| Treated | 1.503 | 1.752 | 1.539 |
| Treated | 1.720 | 2.271 | 1.759 |
| Treated | 1.875 | 2.175 | 1.884 |
| Avg. | 1.512 | 1.894 | 1.600 |
| % of 0/0 | 390 | 220 | 192 |

Interaction of L-alanine and Na-Pyruvate on in vivo AHAS activity in excised leaf discs from newest emerging leaf of canola. Incubated in assay solution for 14 hours. (All assay solutions contained 10 mM Phosphate buffer, pH 6, 500 μM CPCA, and 10% Murashige and Skoog mineral salts.)

TABLE 8

| Alanine | Pyruvate | |
|---|---|---|
| (%) | (mM) | % of No Alaninor Pyruvate |
| 0.5 | 0 | 144 |
| 1 | 0 | 174 |
| 0 | 25 | 110 |
| 0 | 50 | 139 |
| 0.5 | 25 | 188 |
| 0.5 | 50 | 196 |
| 1 | 25 | 238 |
| 1 | 50 | 207 |

AHAS Assay of Resistant and Non-Resistant Crops

For the experiments, plants are grown from seed in soil in a greenhouse. The experiments used wild type and imidazolinone resistant varieties of maize and wheat.

For the assay, a microtiter plate is set up with 100 ml of one of the following solutions in each well:
Solution A: 10 mM phosphate buffer (pH 4.4), 10% prepared Murashige and Skoog basal mineral salt stock solution, 0.5% (w/v) alanine, 50 mM pyruvate salt, and 0.5 mM CPCA (cyclopropyldicarboxylic acid);
Solution B: Solution A+0.0025 mM imazamox;
Solution C: Solution A without 0.5 mM CPCA

Plant material is excised from the newest emerging leaf for wheat or rapidly growing stem for maize from the test plant and placed in one of the microtiter plates. Usually, 3 to 5 mm slices are cut from the newest emerging leaf or stem. At least 3 same-length slices per plant are cut. One disc is put in a well with Solution A, one in a well with Solution B, and the last one in a well with Solution C.

After all wells in the microtiter plate contain plant material, the plate is covered with a plate cover and is placed under continuous fluorescent lights for 14 to 18 hours. The incubation is stopped by placing the plate in a −20° C. freezer. After tissue and solutions freeze, the plate is thawed at room temperature, and 25 mL of 5% sulfuric acid is added to each well. After incubation at 60° C. for 15 minutes, 150 mL of a freshly prepared mixture of α-naphthol (2.5%, w/v) and creatine (0.25%, w/v) in 2 N NaOH are added to each well and the plate is incubated at 60° C. for 15 minutes.

A 190 μl solution from each well is transferred to a new plate, and OD530 on microtiter plate is readed by a spectrophotometer.

AHAS activity of each plant is determined by the following formula:
(OD530 of well B−OD530 of well C)/(OD530 of well A−OD530 of well C).

The results are given in table 9

TABLE 9

| Imidazolinone | Maize | | Wheat | |
|---|---|---|---|---|
| resistant | | Relative | | Relative |
| types | | AHAS activity | | AHAS activity |
| of crops | Variety | (%)* | Variety | (%)* |
| Non-resistance | Pioneer | 5.8 | Riband | 1.5 |
| | 3514 | | | |
| Moderate- | Garst | 45.2 | CV 9804 | 22.4 |
| resistance | 8541 IT | | | |
| High-resistance | Pioneer | 67.3 | Teal 15A | 57.7 |
| | 3395 IR | | | |

*Relative AHAS activity = (OD of 2.5 mM imazamox and CPCA treatment − OD of the background)/(OD of CPCA treatment alone − OD of the background), average of four plants.

Replicate

| | | |
|---|---|---|
| 1 | 2 | 3 |

NH₄OH (50 mM)

| | | | |
|---|---|---|---|
| 0/0 | 0.618 | 0.799 | 0.885 |
| Treated | 0.604 | 0.880 | 1.386 |
| Treated | 0.522 | 0.866 | 1.177 |
| Treated | 1.003 | 0.953 | 1.845 |
| Avg. | 0.697 | 0.886 | 1.386 |
| % of 0/0 | 113 | 111 | 157 |

Pyruvate (100 mM)

| | | | |
|---|---|---|---|
| 0/0 | 1.128 | 1.132 | 1.040 |
| Treated | 1.526 | 1.848 | 1.972 |
| Treated | 1.469 | 1.846 | 2.235 |
| Treated | 1.725 | 1.796 | 2.100 |
| Avg. | 1.510 | 1.730 | 1.950 |
| % of 0/0 | 134 | 153 | 188 |

Pyr & NH₄OH (50 mM each)

| | | | |
|---|---|---|---|
| 0/0 | 0.388 | 0.861 | 0.835 |
| Treated | 1.503 | 1.752 | 1.539 |
| Treated | 1.720 | 2.271 | 1.759 |
| Treated | 1.875 | 2.175 | 1.884 |
| Avg. | 1.512 | 1.894 | 1.600 |
| % of 0/0 | 390 | 220 | 192 |

Table 8

Interaction of L-alanine and Na-Pyruvate on in vivo AHAS activity in excised leaf discs from newest emerging leaf of canola. Incubated in assay solution for 14 hours. (All assay solutions contained 10 mM Phosphate buffer, pH 6, 500 μM CPCA, and 10% Murashige and Skoog mineral salts.)

| Alanine | Pyruvate | |
|---|---|---|
| (%) | (mM) | % of No Alaninor Pyruvate |
| 0.5 | 0 | 144 |
| 1 | 0 | 174 |
| 0 | 25 | 110 |
| 0 | 50 | 139 |
| 0.5 | 25 | 188 |
| 0.5 | 50 | 196 |
| 1 | 25 | 238 |
| 1 | 50 | 207 |

1. A method for determining whether a plant is resistant to an acetohydroxyacid synthase inhibitor comprising:
treating a first part taken from the plant with a acetohydroxyacid synthase inhibitor, a ketol-acid reductoisomerase inhibitor and a first external supplement comprising alanine, ammonium hydroxide, asparagine, pyruvic acid or a salt thereof or a combination of any of the preceding compounds; and
measuring a first amount of acetohydroxybutyrate or acetolactate accumulated by said first part.

2. The method of claim 1 further comprising:
treating a second part taken from the plant with a ketol-acid reductoisomerase inhibitor and a second external supplement comprising alanine, ammonium hydroxide, asparagine, pyruvic acid or a salt thereof, or a combination of any of the proceeding compounds; and
determining a second amount of acetohydroxybutyrate or acetolactate accumulated by said second part; and
comparing the first amount accumulated by said first part with the second amount accumulated by said second part.

3. A method for determining whether a test-compound inhibits acetohydroxyacid synthase comprising:
treating a first part taken from the plant with a test-compound, a ketol-acid reductoisomerase inhibitor and an external supplement comprising alanine, ammonium hydroxide, asparagine, and pyruvic acid or a salt thereof, or a combination of any of the preceding compounds; and
measuring a first amount of acetohydroxybutyrate acetolactate accumulated by said first part.

4. The method to of claim 3 further comprising:
treating a second part taken from the plant with a ketol-acid reductoisomerase inhibitor and a second external supplement comprising alanine, ammonium hydroxide, asparagine, and pyruvic acid or a salt thereof, or a combination of any of the preceding compounds; and
determining a second amount of acetohydroxybutyrate or acetolactate accumulated by said second part; and
comparing the first amount of accumulated by said first part with the second amount accumulated by said second part.

5. The method of claim 1, wherein the first part is selected from an actively growing region of said plant.

6. The method of claim 1, wherein the first part is taken from a basipetal region of a blade of the plant.

7. The method of claim 2, wherein the plant has a blade with a first portion and a second portion separated by a midvein, wherein the first part is taken from the first portion and the second part is taken from the second portion.

8. The method of claim 1, wherein the first external supplement is selected from the group consisting of alanine, ammonium hydroxide, pyruvic acid, a salt of alanine, a salt of pyruvic acid and mixtures thereof.

9. The method of claim 8, wherein the first external supplement is alanine and pyruvic acid or a salt thereof.

10. The method of claim 9, wherein the alanine and pyruvic acid or salt thereof are present at a ratio of about 8:1 to 1:50.

11. The method of claim 8, wherein the first external supplement is ammonium hydroxide and pyruvic acid or a salt thereof.

12. The method of claim 11, wherein the ammonium hydroxide and pyruvic acid or a salt thereof are present at a ratio of about 8:1 to 1:50.

13. The method of claim 1, wherein the first external supplement for treating said first part is present at about 0.2 to less than 5 percent by weight of a sum of the weights of the first part the acetohydroxyacid synthase inhibitor, the ketol-acid reductoisomerase inhibitor and the first external supplement.

14. The method of claim 1, wherein the ketol-acid reductoisomerase inhibitor is selected from the group consisting of (dimethylphosphinyl) glycolic acid, 2-(dimethylphosphinoyl)-2-hydroxyacetic acid, a sodium N-hydroxy-N-(C₁-C₆ alkyl or C₃-C₇ cycloalkyl) oxamate, sodium N-hydroxy-N-aralkyloxamate, 2-methylphosphinoyl-2-hydroxyacetic acid, N-hydroxy-N-isopropyloxamate, a monomethyl and monoethyl ester of 1,1-cyclopropanedicarboxylic acid, ethylenemalonic acid or a mono or disalt thereof, and combinations thereof.

15. The method of claim 14, wherein the sodium-N-hydroxy-N-(C₁-C₆ alkyl) oxamate is sodium N-hydroxy-N-isopropyloxamate.

16. The method of claim 14, wherein the sodium N-hydroxy-N-aralkyloxamate is sodium N-hydroxy-N-benzyloxamate.

17. The method of claim 2, wherein the second part is selected from an actively growing region of said plant.

18. The method of claim 2, wherein the second part is taken from a basipetal region of a blade of the plant.

19. The method of claim 7, wherein the first and second portions are taken from approximately equal and opposite locations of said blade.

20. The method of claim 2, wherein the second external supplement is selected from the group consisting of alanine, ammonium hydroxide, pyruvic acid, a salt of alanine, a salt of pyruvic acid and mixtures thereof.

21. The method of claim 20, wherein the second external supplement is alanine and pyruvic acid or a salt thereof.

22. The method of claim 21, wherein the alanine and pyruvic acid or salt thereof are present at a ratio of about 8:1 to 1:50.

23. The method of claim 2, wherein the second external supplement is ammonium hydroxide and pyruvic acid or a salt thereof.

24. The method of claim 23, wherein the ammonium hydroxide and pyruvic acid or a salt thereof are present at a ratio of about 8:1 to 1:50.

25. The method of claim 2, wherein the second external supplement for treating said second part is present in an amount of about 0.2 to less than 5 percent by weight of a sum of the weights of the second part, the acetohydroxyacid synthase inhibitor, the ketol-acid reductoisomerase inhibitor and the second external supplement.

26. The method of claim 25, wherein the ketol-acid reductoisomerase inhibitor is selected from the group consisting of (dimethylphosphinyl) glycolic acid, 2-(dimethylphosphinoyl)-2-hydroxyacetic acid, a sodium N-hydroxy-N-(C₁-C₆ alkyl or C₃-C₇ cycloalkyl) oxamate, sodium N-hydroxy-N-aralkyloxamate, 2-methylphosphinoyl-2-hydroxyacetic acid, N-hydroxy-N-isopropyloxamate, a monomethyl or monoethyl ester of 1,1-cyclopropanedicarboxylic acid, an ethylenemalonic acid or a mono or disalt thereof, and combinations thereof.

27. The method of claim 26, wherein the sodium-N-hydroxy-N-(C₁-C₆ alkyl) oxamate is sodium N-hydroxy-N-isopropyloxamate.

28. The method of claim 26, wherein the sodium N-hydroxy-N-aralkyloxamate is sodium N-hydroxy-N-benzyloxamate.

29. The method of claim 2, wherein the first external supplement and the second external supplement are the same.

30. The method of claim 2, wherein the first external supplement and the second external supplement are different.

31. The method of claim 3, wherein the first part is selected from an actively growing region of said plant.

32. The method of claim 4, wherein the second part is selected from an actively growing region of said plant.

33. The method of claim 3, wherein the first part is taken from a basipetal region of a blade of the plant.

34. The method of claim 4, wherein the second part is taken from a basipetal region of a blade of the plant.

35. The method of claim 4, wherein the plant has a blade with a first portion and a second portion separated by a midvein, wherein the first part is taken from the first portion and the second part is taken from the second portion.

36. The method of claim 35, wherein the first and second portions are taken from approximately equal and opposite locations of said blade.

37. The method of claim 3, wherein the first external supplement is selected from the group consisting of alanine, ammonium hydroxide, pyruvic acid, a salt of alanine, a salt of pyruvic acid and mixtures thereof.

38. The method of claim 37, wherein the first external supplement is alanine and pyruvic acid or a salt thereof.

39. The method of claim 38, wherein the alanine and pyruvic acid or salt thereof are present at a ratio of about 8:1 to 1:50.

40. The method of claim 37, wherein the first external supplement is ammonium hydroxide and pyruvic acid or a salt thereof.

41. The method of claim 40, wherein the ammonium hydroxide and pyruvic acid or a salt thereof are present at a ratio of about 8:1 to 1:50.

42. The method of claim 3, wherein the first external supplement for treating said first part is present at about 0.2 to less than 5 percent by weight of a sum of the weights of the first part, the acetohydroxyacid synthase inhibitor, the ketol-acid reductoisomerase inhibitor and the first external supplement.

43. The method of claim 3, wherein the ketol-acid reductoisomerase inhibitor is selected from the group consisting of (dimethylphosphinyl) glycolic acid, 2-(dimethylphosphinoyl)-2-hydroxyacetic acid, a sodium N-hydroxy-N-(C₁-C₆ alkyl or C₃-C₇ cycloalkyl) oxamate, sodium N-hydroxy-N-aralkyloxamate, 2-methylphosphinoyl-2-hydroxyacetic acid, N-hydroxy-N-isopropyloxamate, a monomethyl and monoethyl ester of 1,1-cyclopropanedicarboxylic acid, ethylenemalonic acid or a mono or disalt thereof, and combinations thereof.

44. The method of claim 43, wherein the sodium-N-hydroxy-N-(C₁-C₆ alkyl) oxamate is sodium N-hydroxy-N-isopropyloxamate.

45. The method of claim 43, wherein the sodium N-hydroxy-N-aralkyloxamate is sodium N-hydroxy-N-benzyloxamate.

46. The method of claim 4, wherein the second external supplement is selected from the group consisting of alanine, ammonium hydroxide, pyruvic acid, a salt of alanine, a salt of pyruvic acid and mixtures thereof.

47. The method of claim 46, wherein the second external supplement is alanine and pyruvic acid or a salt thereof.

48. The method of claim 47, wherein the alanine and pyruvic acid or salt thereof are present at a ratio of about 8:1 to 1:50.

49. The method of claim 46, wherein the second external supplement is ammonium hydroxide and pyruvic acid or a salt thereof.

50. The method of claim 49, wherein the ammonium hydroxide and pyruvic acid or a salt thereof are present at a ratio of about 8:1 to 1:50.

51. The method of claim 4, wherein the second external supplement for treating said second part is present in an amount of about 0.2 to less than 5 percent by weight of a sum of the weights of the second part, the acetohydroxyacid synthase inhibitor, the ketol-acid reductoisomerase inhibitor and the second external supplement.

52. The method of claim 51, wherein the ketol-acid reductoisomerase inhibitor is selected from the group consisting of (dimethylphosphinyl) glycolic acid, 2-(dimethylphosphinoyl)-2-hydroxyacetic acid, a sodium N-hydroxy-N-(C₁-C₆ alkyl or C₃-C₇ cycloalkyl) oxamate, sodium N-hydroxy-N-aralkyloxamate, 2-methylphosphinoyl-2-hydroxyacetic acid, N-hydroxy-N-isopropyloxamate, a monomethyl or monoethyl ester of 1,1-cyclopropanedicarboxylic acid, an ethylenemalonic acid or a mono or disalt thereof, and combinations thereof.

53. The method of claim 52, wherein the sodium-N-hydroxy-N-(C₁-C₆ alkyl) oxamate is sodium N-hydroxy-N-isopropyloxamate.

54. The method of claim 52, wherein the sodium N-hydroxy-N-aralkyloxamate is sodium N-hydroxy-N-benzyloxamate.

55. The method of claim 4, wherein the first external supplement and the second external supplement are the same.

56. The method of claim 4, wherein the first external supplement and the second external supplement are different.

57. The method of claim 4, further comprising treating said second part with said test compound.
